(19) United States
(12) Patent Application Publication
(10) Pub. No.: US 2025/0344625 A1
(43) Pub. Date: Nov. 13, 2025

(54) SYSTEM AND METHOD FOR ESTIMATING CROP WATER REQUIREMENT USING MULTI-SENSOR DATA FUSION

(71) Applicant: Tata Consultancy Services Limited, Mumbai (IN)

(72) Inventors: Suryakant Ashok SAWANT, Pune (IN); Jay Prakash KUMAR, Bangalore (IN); Jayantrao MOHITE, Thane (West) (IN); Ankur PANDIT, Indore (IN); Srinivasu PAPPULA, Hyderabad (IN)

(73) Assignee: Tata Consultancy Services Limited, Mumbai (IN)

(21) Appl. No.: 19/090,545

(22) Filed: Mar. 26, 2025

(30) Foreign Application Priority Data
May 7, 2024 (IN) 202421036177

(51) Int. Cl. A01B 79/00; G01S 13/90; G01S 13/95; G01W 1/02; G01W 1/14

(52) U.S. Cl. CPC A01B 79/005; G01S 13/90; G01S 13/955; G01W 1/02; G01W 1/14

(57) ABSTRACT

This disclosure relates generally to system and method for estimating crop water requirement using multi-sensor data fusion. Increasing global population is imparting pressure on both agriculture for food demand and limited freshwater resources for consumption. Estimating crop water requirement reduces water demand for crop production. The method divides soil and crop into multiple vertical and horizontal profiles to estimate water balance thereby reducing the errors in estimation of crop water requirement. Additionally, the method has capability to interlink the multiple data sets such as satellite based earth observations, weather observations from IoT sensors, Weather forecasts from global circulation models, and crop knowledge base for crop water requirement estimation. The method is based on spatio-temporal modeling for multi-layer crop and soil water balance and helps to generate the additional insights on crop water requirement like moisture at different levels in soil profile, crop canopy growth at different locations.

### PRIORITY CLAIM

This U.S. patent application claims priority under 35 U.S.C. § 119 to: Indian Patent Application 202421036177, filed on May 7, 2024. The entire contents of the aforementioned application are incorporated herein by reference.

### TECHNICAL FIELD

The disclosure herein generally relates to estimation of crop water requirement, and, more particularly, to system and method for estimating crop water requirement using multi-sensor data fusion.

### BACKGROUND

Agriculture, industry, and urban areas are competing for available water resources. Increasing global population is imparting pressure on both agriculture for food demand and limited freshwater resources for consumption. Crop water requirements for crops are found to estimate the total quantity of water required for the crops for their full growth throughout their growing period. The water requirement of every crop is different, and it also depends upon the weather conditions of that particular area. With the advent of accurate global weather forecast models, low orbit earth observation satellites and precision irrigation technologies have opened opportunities to understand the crop growth conditions.

Crop water requirement is a function of crop characteristics, crop management, soil, and weather which is defined as depth of water required to meet the water consumed through crop metabolic activities such as evaporation and transpiration. Better crop water management results in healthy crop, growing in fields under non-restricting soil conditions including soil water and fertility, and achieving full production potential under the given growing environment.

Variations in local weather conditions, crop management practices and variable soil conditions pose challenges to accurate estimation of crop water requirement. Accurate estimate of crop water requirement leads to reduction in water and energy use. The scalability of current crop water requirement estimation models is limited by availability of accurate weather observations, weather forecasts, information about the current crop growth phase and dynamics of soil water balance. Continued evolution in the field level agricultural cropping practices and enhancements is the desired outcome of the present challenges.

### SUMMARY

Embodiments of the present disclosure present technological improvements as solutions to one or more of the above-mentioned technical problems recognized by the inventors in conventional systems. For example, in one embodiment, a method for field level crop water requirement estimation is provided. The system includes receiving from a field a set of inputs comprising a IoT sensor data, one or more crop characteristics, one or more agro-meteorological weather data of the field, one or more satellite earth observation data covering the field, one or more soil properties of the field, a historical irrigation data, and a weather forecast data. The one or more agro-meteorological weather data includes a temperature, a relative humidity, a wind speed, a wind direction, an effective rainfall information, and a solar radiation information. The IoT sensor data includes a soil moisture, a soil temperature, and an air temperature. Further, the set of inputs are utilized for estimation of (i) a reference crop evapotranspiration (ET) using the one or more agro-meteorological weather data and the IoT sensor data (ii) a crop ET under standard conditions (ET_{c}) using a crop coefficient (iii) a crop ET under non-standard conditions (ETₚₘ) using the crop coefficient and a soil factor (iv) a remote sensing based evapotranspiration (ETᵣₛ) using the one or more satellite earth observation data covering the field and (v) a field evapotranspiration ET. Here, the ET filed is an ensemble of the remote sensing based evapotranspiration and the crop ET under non-standard conditions. Then, a runoff is estimated using a curve number method based on the one or more agro-meteorological weather, and the one or more satellite earth observation data covering the field.

Further, a soil water balance of the field is computed using the field level evapotranspiration (ET_{field}), the runoff, the effective rainfall information, the IoT sensor data, the one or more crop characteristics and the one or more soil properties. Then, an inverse modelling approach is applied to estimate the one or more soil properties and the one or more crop characteristics. The one or more soil properties includes a field capacity, and the one or more crop characteristics includes the crop coefficient.

Furthermore, an ensemble-based field soil moisture is estimated using the soil water balance, and a soil moisture obtained using the IoT sensor data and a soil moisture estimated using a synthetic aperture radar satellite data. Finally, crop water requirement of the field is estimated using (i) the field soil moisture and (ii) a plurality of irrigation scheduling parameters comprising an irrigation interval, a depth of irrigation, a percent allowable depletion based on depletion coefficient.

In another aspect, a method for estimating crop water requirement using multi-sensor data fusion is provided. The method includes receiving from a field a set of inputs comprising a IoT sensor data, one or more crop characteristics, one or more agro-meteorological weather data of the field, one or more satellite earth observation data covering the field, one or more soil properties of the field, a historical irrigation data, and a weather forecast data. The one or more agro-meteorological weather data includes a temperature, a relative humidity, a wind speed, a wind direction, an effective rainfall information, and a solar radiation information. The IoT sensor data includes a soil moisture, a soil temperature, and an air temperature. Further, the set of inputs are utilized for estimation of (i) a reference crop evapotranspiration (ET) using the one or more agro-meteorological weather data and the IoT sensor data (ii) a crop ET under standard conditions (ET_{c}) using a crop coefficient (iii) a crop ET under non-standard conditions ETₚₘ) using the crop coefficient and a soil factor (iv) a remote sensing based evapotranspiration using the one or more satellite earth observation data covering the field and (v) a field evapotranspiration ETET_{field}). Here, the ET field is an ensemble of the remote sensing based evapotranspiration and the crop ET under non-standard conditions. Then, a runoff is estimated using a curve number method based on the one or more agro-meteorological weather, and the one or more satellite earth observation data covering the field.

Further, a soil water balance of the field is computed using the field level evapotranspiration (ET_{field}), the runoff, the effective rainfall information, the IoT sensor data, the one or more crop characteristics and the one or more soil properties. Then, an inverse modelling approach is applied to estimate the one or more soil properties and the one or more crop characteristics. The one or more soil properties includes a field capacity, and the one or more crop characteristics includes the crop coefficient.

Furthermore, an ensemble-based field soil moisture is estimated using the soil water balance, and a soil moisture estimated using the IoT sensor data and a soil moisture estimated using a synthetic aperture radar satellite data. Finally, crop water requirement of the field is estimated using (i) the field soil moisture and (ii) a plurality of irrigation scheduling parameters comprising an irrigation interval, a depth of irrigation, a percent allowable depletion based on depletion coefficient.

In yet another aspect, there are provided one or more non-transitory machine-readable information storage mediums comprising one or more instructions, which when executed by one or more hardware processors causes a method for estimating crop water requirement using multi-sensor data fusion. The method includes receiving from a field a set of inputs comprising a IoT sensor data, one or more crop characteristics, one or more agro-meteorological weather data of the field, one or more satellite earth observation data covering the field, one or more soil properties of the field, a historical irrigation data, and a weather forecast data. The one or more agro-meteorological weather data includes a temperature, a relative humidity, a wind speed, a wind direction, an effective rainfall information, and a solar radiation information. The IoT sensor data includes a soil moisture, a soil temperature, and an air temperature. Further, the set of inputs are utilized to (i) a reference crop evapotranspiration (ET) using the one or more agro-meteorological weather data and the IoT sensor data (ii) a crop ET under standard conditions (ET_{c}) using a crop coefficient (iii) a crop ET under non-standard conditions (ETₚₘ) using the crop coefficient and a soil factor (iv) a remote sensing based evapotranspiration (ETᵣₛ) using the one or more satellite earth observation data covering the field and (v) a field evapotranspiration ET (ET_{field}). Here, the ET filed is an ensemble of the remote sensing based evapotranspiration and the crop ET under non-standard conditions. Then, a runoff is estimated using a curve number method based on the one or more agro-meteorological weather, and the one or more satellite earth observation data covering the field.

Further, a soil water balance of the field is computed using the field level evapotranspiration (ET_{field}), the runoff, the effective rainfall information, the IoT sensor data, the one or more crop characteristics and the one or more soil properties. Then, an inverse modelling approach is applied to estimate the one or more soil properties and the one or more crop characteristics. The one or more soil properties includes a field capacity, and the one or more crop characteristics includes the crop coefficient.

Furthermore, an ensemble-based field soil moisture is estimated using the soil water balance, and a soil moisture estimated using the IoT sensor data and a soil moisture estimated using a synthetic aperture radar satellite data. Finally, crop water requirement of the field is estimated using (i) the field soil moisture and (ii) a plurality of irrigation scheduling parameters comprising an irrigation interval, a depth of irrigation, a percent allowable depletion based on depletion coefficient.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles:

FIG. 1 is a schematic view of an environment where a system is deployed for estimation of crop water requirement using multi-sensor data fusion, in accordance with some embodiments of the present disclosure.

FIG. 2 is a functional block diagram of a system for estimation of crop water requirement, in accordance with some embodiments of the present disclosure.

FIG. 3 (collectively referred as FIG. 3A and FIG. 3B) depicts a use case example for estimating crop water requirement using the system of FIG. 2, in accordance with some embodiments of the present disclosure, in accordance with some embodiments of the present disclosure.

FIG. 4 (collectively referred as FIG. 4A and FIG. 4B) is a flow diagram illustrating a method for FIG. 2 depicts process flow of the system for estimating crop water requirement, in accordance with some embodiments of the present disclosure.

FIG. 5 is a visual representation of variation of air and water in soil profile at various time steps using the system of FIG. 2, in accordance with some embodiments of the present disclosure, in accordance with some embodiments of the present disclosure.

FIG. 6 is a visual representation of soil water balance using the system of FIG. 2, in accordance with some embodiments of the present disclosure, in accordance with some embodiments of the present disclosure.

### DETAILED DESCRIPTION

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the scope of the disclosed embodiments.

### Glossary

Rainfall: Water received by precipitation rainfall or snow.

Irrigation: Water provided using irrigation system using methods like flood, surface drip, sub-surface drip, sprinkler.

Evapotranspiration (ET): Estimate of Evaporation and Transpiration of selected crop in the field. ET is divided into two types refence ET and crop ET.

Runoff: Excess water that moves out from fields as flow in drains. Runoff is the part of the water cycle that flows over the land as surface water rather than being absorbed into groundwater or evaporating. It appears in uncontrolled surface streams, rivers, drains, or sewers. Factors that affect runoff include the amount of rainfall, permeability, vegetation, and the slope of the land.

Percolation: Excess water moving out of soil layer and can be available as capillary rise.

Deep percolation: Excess water moving out of soil layer and un-available to the crop.

Capillary rise: Vertical movement of the water through capillary rise.

Field Capacity: Field capacity is the amount of water that is retained in the soil after excess water has drained away and the rate of downward movement has decreased. This usually takes place 2-3 days after rain or irrigation in pervious soils of uniform structure and texture.

Wilting Point: The wilting point is the soil moisture content at which plants can no longer absorb water from the soil. This occurs when the water potential in the soil is lower than the water potential in the plant roots.

Total Available Water (TAW): Total water stored in soil profile at field capacity and available for crop use.

Depletion Coefficient: Depletion coefficient is dimensionless number used to control availability of water in soil profile.

Readily Available Water/profile moisture: Lower level of moisture available in soil profile for crop use.

Available profile moisture: moisture available in soil profile at given time.

Irrigation Event: Day of providing the irrigation with amount of irrigation.

Soil Depth, Root Zone Depth: Depth of soil layer, Effective soil depth available for crop root growth.

Crop Coefficient: A crop coefficient is a dimensionless number that represents the ratio of the actual crop Evapotranspiration ET_{c} to the reference Evapotranspiration ETₒ. ET_{c} is the amount of water that is lost from a crop through transpiration and evaporation, while ETₒ is the amount of water that would be lost from a well-watered reference crop, such as grass, under the same environmental conditions. Crop coefficients vary depending on the crop type, the stage of crop growth, and the environmental conditions. For example in Tea Crop Coefficient is function of harvest, pruning and shade tree density.

Water is a crucial or important factor for crop production. More than 80% of water resources have been exploited for irrigation. Water requirement of crops refers to the amount of water that is needed by a particular crop during its growth cycle to produce an optimum yield. The water requirement varies from crop to crop and is affected by various factors such as the stage of crop growth, weather conditions, soil type, and the availability of water. Understanding the water requirement of crops is crucial for farmers and agricultural experts to implement effective water management strategies and ensure sustainable crop production.

Crop water requirements (CWR) refers to the amount of water required to compensate for evapotranspiration losses from a cropped field during a specified period. The concept of crop water requirements has become important with the development of large engineering works when it was necessary to estimate the water volumes to be supplied to newly irrigated areas. Crop water requirement is required to meet the water loss through evapotranspiration of a disease-free crop growing in fields under variable soil conditions, including soil water and fertility status, and achieving its full grain production potential under the given environmental conditions (including soil water and fertility status). Identifying water requirements and crop coefficients is critical for irrigation scheduling and agricultural water management in field management.

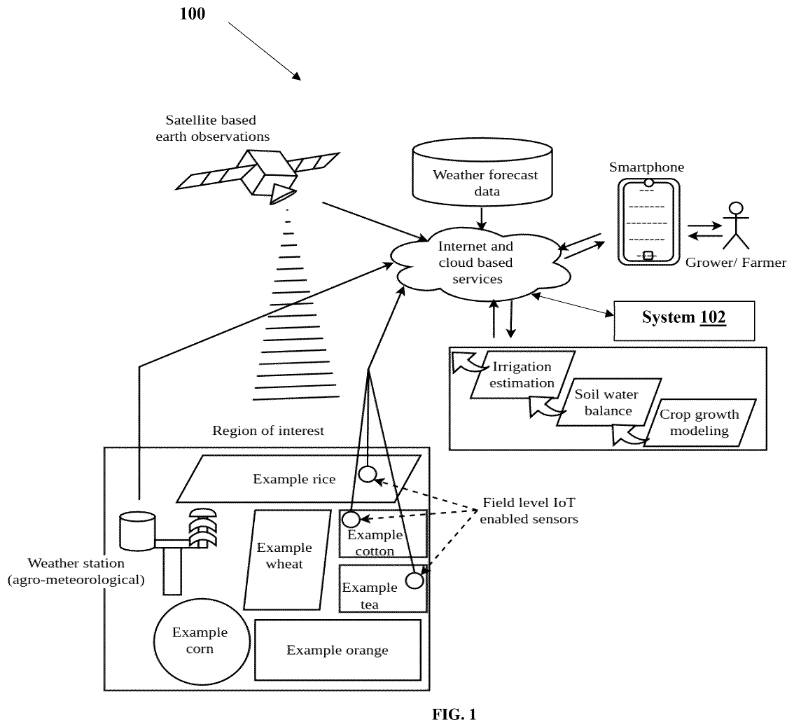

Embodiments herein provide a system and method for estimating crop water requirement using multi-sensor data fusion. The system may be alternatively referred as a water requirement estimation system. The system is a multi-layered crop and soil water balance modeling using a plurality of sensors. The method of the present disclosure divides both soil and crop into multiple vertical and horizontal profiles to estimate water balance thereby reducing the errors in estimation of crop water requirement. Observations from multiple sources such as satellite-based earth observations, regional weather observations from agro-meteorological systems, field level weather observations from IoT sensors, weather forecasts from global circulation models, and crop knowledge base is used for crop water requirement estimation. The method is based on spatio-temporal modeling for multi-layer crop and soil water balance and helps to generate the additional insights on crop water requirement like moisture at different levels in soil profile, crop canopy growth at different locations. The disclosed system is further explained with the method as described in conjunction with FIG. 1 to FIG. 6 below.

Referring now to the drawings, and more particularly to FIG. 1 through FIG. 6, where similar reference characters denote corresponding features consistently throughout the figures, there are shown preferred embodiments and these embodiments are described in the context of the following exemplary system and/or method.

FIG. 1 is a schematic view of an environment where a system is deployed for estimation of crop water requirement using multi-sensor data fusion, in accordance with some embodiments of the present disclosure.

The FIG. 1 depicts a plurality of fields, each field also referred to as agriculture or horticulture field of interest. Satellite based sensor captures agriculture or horticulture field data for an area of coverage. The coverage area includes the agriculture field comprising one or more growing crops, for example sugarcane, crop, wheat, rice, cotton, tea, orange, and the like for cultivating different crops. The system 102, with known in the art techniques tag the geographical location of agriculture crop fields to identify boundaries of each field of interest. Each field may have IoT enabled sensors to capture information such as soil moisture, soil temperature, air temperature, and the like. Agro-meteorological sensors capture weather data of the field under coverage transmitting the sensor information via internet to cloud based services storage. The region having agro-meteorological weather station has sensors that measure weather parameters such as temperature, relative humidity, wind speed, wind direction, rainfall, solar radiation, and the like. The weather parameters include maximum temperature and minimum temperature, maximum relative humidity, minimum relative humidity wind speed, solar radiation, and the effective rainfall. The field sensor data includes the soil moisture. The soil parameters include field capacity, wilting point, and total available water for that crop based on root zone depth. The crop parameters include the crop coefficient, crop growth stage, root zone depth, depletion coefficient and the crop area.

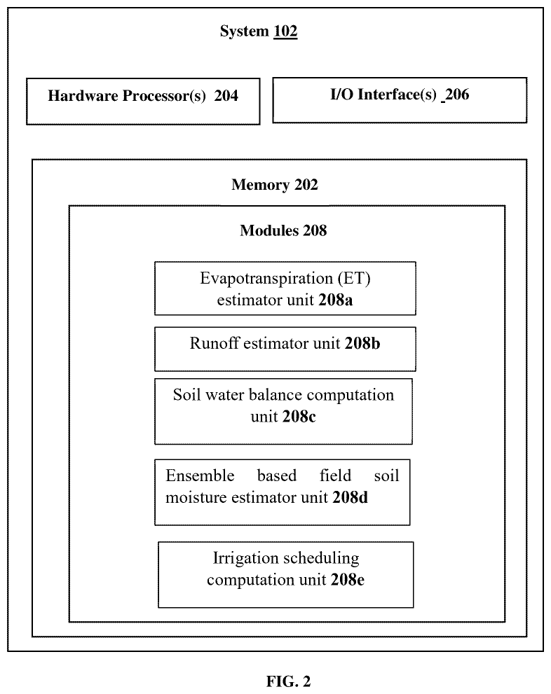

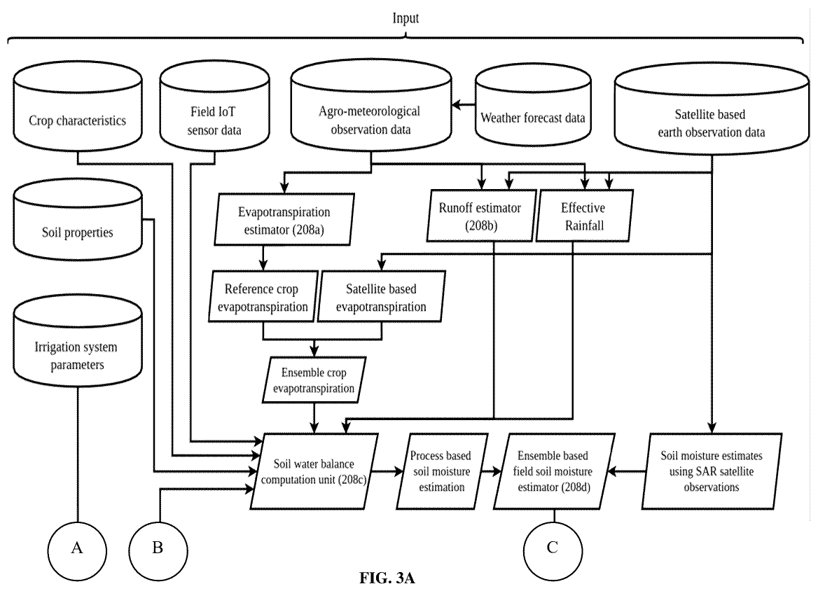

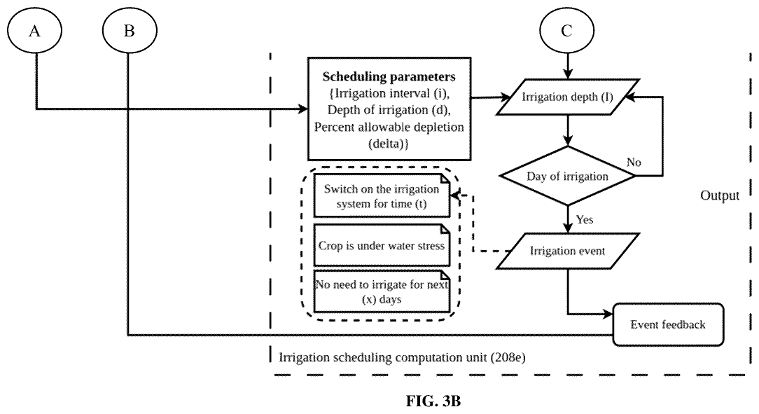

The data captured from the IoT based sensors and the agro-meteorological sensors are transmitted to cloud based data store or service which is further used by multiple stakeholders. It is noted each field may have or may not have sensor(s). Each field may not have the sensor deployed then regional agro-meteorological observations and satellite based observations utilized in the method. Additionally, each field has onsite deployed sensors that directly communicate with a cloud server and associated mobile devices through which respective farmers or landowners update information related to various events taking place on the agriculture field of interest into the cloud server. The satellite or drone based earth observation data is collected in multi-spectral, hyperspectral, thermal and Synthetic Aperture Radar (SAR) sensors. The earth observation data is available in open source and commercial modes. The earth observations data from multi-spectral or hyperspectral sensors is used for calculation of vegetation indices, whereas SAR data is used for calculation of soil moisture (Pandit et al., 2022). Weather forecast data is obtained from process based global circulation models. The weather forecast data includes parameters such as temperature, humidity, precipitation (rainfall), solar radiation, etc. The weather forecasts of short and medium term (about seven to fifteen days) are also used for the crop water requirement analysis. The geospatial boundary of each field, crop management practices, information related to irrigation system is collected using mobile or web application installed on the Smartphone or tablet or computing device (laptop or personal computer) of the user (farmer, or stakeholder). The region specific information about soil that includes soil type, soil texture, soil depth and elevation of the field is obtained using regional soil database. The system also records the information on crops grown in the region, neighboring crop fields, weather data, and harvesting patterns and the like. The system 102 is further explained with respect to FIG. 2 through FIG. 6 and method depicted in flow diagram of FIG. 3.

FIG. 2 is a functional block diagram of a system for estimation of crop water requirement, in accordance with some embodiments of the present disclosure.

In an embodiment, the system 102 includes a processor(s) 204, communication interface device(s), alternatively referred as input/output (I/O) interface(s) 206, and one or more data storage devices or a memory 202 operatively coupled to the processor(s) 204. The system 102 with one or more hardware processors is configured to execute functions of one or more functional blocks of the system 102.

Referring to the components of the system 102, in an embodiment, the processor(s) 204, can be one or more hardware processors 204. In an embodiment, the one or more hardware processors 204 can be implemented as one or more microprocessors, microcomputers, microcontrollers, digital signal processors, central processing units, state machines, logic circuitries, and/or any devices that manipulate signals based on operational instructions. Among other capabilities, the one or more hardware processors 204 are configured to fetch and execute computer-readable instructions stored in the memory 202. In an embodiment, the system 102 can be implemented in a variety of computing systems including laptop computers, notebooks, hand-held devices such as mobile phones, workstations, mainframe computers, servers, and the like.

The I/O interface(s) 206 can include a variety of software and hardware interfaces, for example, a web interface, a graphical user interface to display the generated target images and the like and can facilitate multiple communications within a wide variety of networks N/W and protocol types, including wired networks, for example, LAN, cable, etc., and wireless networks, such as WLAN, LoRA, cellular and the like. In an embodiment, the I/O interface (s) 206 can include one or more ports for connecting to a number of external devices or to another server or devices.

The memory 202 may include any computer-readable medium known in the art including, for example, volatile memory, such as static random access memory (SRAM) and dynamic random access memory (DRAM), and/or non-volatile memory, such as read only memory (ROM), erasable programmable ROM, flash memories, hard disks, optical disks, and magnetic tapes. In an embodiment, the memory 202 includes a plurality of modules 208 comprising a evapotranspiration estimator 208a, a runoff estimator 208b, a soil water balance computation unit 208c, an ensemble based field soil moisture estimator 208d and an irrigation scheduling computation unit 208e.

The plurality of modules 210 include programs or coded instructions that supplement applications or functions performed by the system 102 for executing different steps involved in the process of estimation of soil water balance, being performed by the system 102. The plurality of modules 210, amongst other things, can include routines, programs, objects, components, and data structures, which performs particular tasks or implement particular abstract data types. The plurality of modules 210 may also be used as, signal processor(s), node machine(s), logic circuitries, and/or any other device or component that manipulates signals based on operational instructions. Further, the plurality of modules 210 can be used by hardware, by computer-readable instructions executed by the one or more hardware processors 104, or by a combination thereof. The plurality of modules 210 can include various sub-modules (not shown).

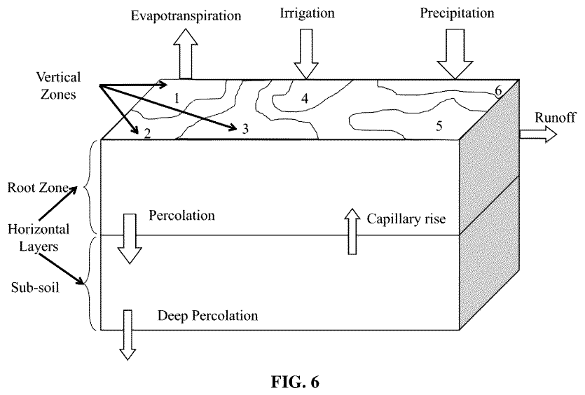

Further, the memory 102 may comprise information pertaining to input(s)/output(s) of each step performed by the processor(s) 104 of the system 102 and methods of the present disclosure. Further, the memory 202 includes a database 208. Although the database 208 is shown internal to the system 102, it will be noted that, in alternate embodiments, the database 208 can also be implemented external to the system 102, and communicatively coupled to the system 102. The data contained within such external database may be periodically updated. For example, new data may be added into the database (not shown in FIG. 2) and/or existing data may be modified and/or non-useful data may be deleted from the database. In one example, the data may be stored in an external system, such as a Lightweight Directory Access Protocol (LDAP) directory and a Relational Database Management System (RDBMS). Functions of the components of the system 102 are now explained with reference to FIG. 2 through steps of flow diagram in FIG. 6.

FIG. 3 (collectively referred as FIG. 3A and FIG. 3B) depicts a use case example for estimating crop water requirement using the system of FIG. 2, in accordance with some embodiments of the present disclosure, in accordance with some embodiments of the present disclosure. In an embodiment, the memory 202 includes a plurality of modules 208 comprising the evapotranspiration estimator 208a, the runoff estimator 208b, the soil water balance computation unit 208c, the ensemble based field soil moisture estimator 208d and the irrigation scheduling computation unit 208e.

The evapotranspiration estimator 208a of the system 100 estimates reference ET, crop ET and crop ET at non-standard conditions or field conditions. The ET rate from a reference surface, not short of water, is called the reference crop ET or reference ET and is denoted as ET_{O}.

The runoff estimator 208b estimates of the system 100 land cover condition using satellite based land use land cover classification and antecedent rainfall observed by weather station.

The soil water balance computation unit 208c of the system 100 computes conceptual multi-layered (horizontal and vertical) model.

The ensemble based field soil moisture estimator 208d of the system 100 dynamically adjusts one or more parameters used for crop water requirement estimation.

The irrigation scheduling computation unit 208e of the system 100 considers entire soil profile. The soil profile comprises of solid, air and water, where the water is absorbed by roots for consumptive use and empty space is filled by air.

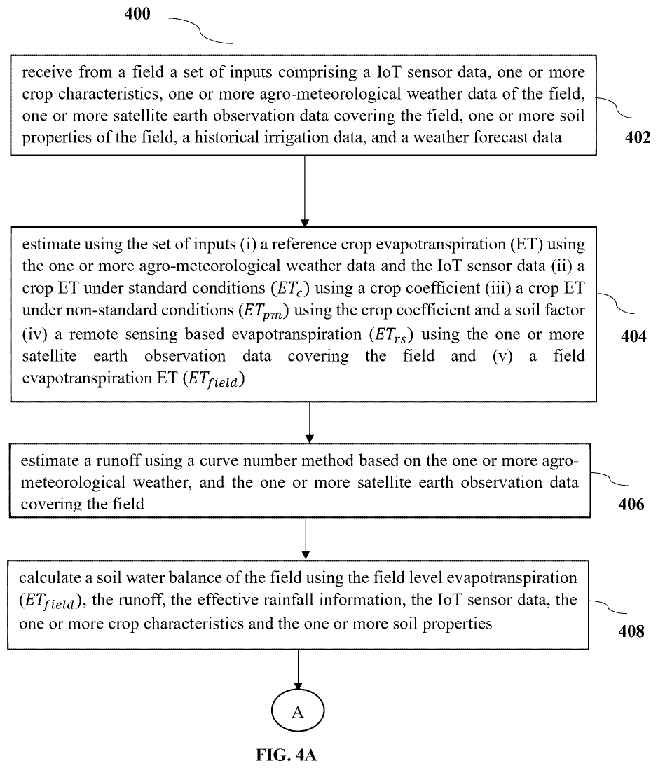

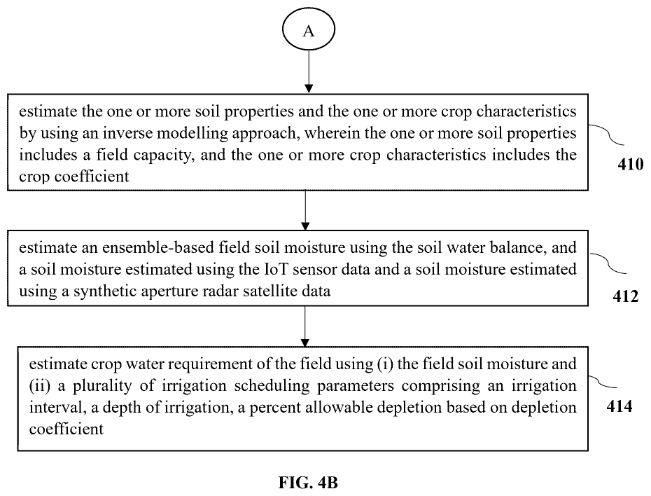

FIG. 4 (collectively referred as FIG. 4A and FIG. 4B) is a flow diagram illustrating a method for FIG. 2 depicts process flow of the system for estimating crop water requirement, in accordance with some embodiments of the present disclosure.

In an embodiment, the system 100 comprises one or more data storage devices or the memory 102 operatively coupled to the processor(s) 104 and is configured to store instructions for execution of steps of a method 400 by the processor(s) or one or more hardware processors 104. The steps of the method 400 of the present disclosure will now be explained with reference to the components or blocks of the system 100 as depicted in FIG. 1 through FIG. 2, and the steps of flow diagram as depicted in FIG. 4. Although process steps, method steps, techniques or the like may be described in a sequential order, such processes, methods, and techniques may be configured to work in alternate orders. In other words, any sequence or order of steps that may be described does not necessarily indicate a requirement that the steps to be performed in that order. The steps of processes described herein may be performed in any order practical. Further, some steps may be performed simultaneously.

Referring to the steps of the method 400, at step 402 a one or more hardware processor is configured to receive from a field a set of inputs comprising a IoT sensor data, one or more crop characteristics, one or more agro-meteorological weather data of the field, one or more satellite earth data covering the field, one or more soil properties of the field, a historical irrigation data, and a weather forecast data.

Referring to an example, where the system 100 receives a request from external users to estimate field level crop water requirement on daily basis. The external user may be farmer, crop grower and the like. Once the request is received, the system 100 collects the set of inputs corresponding to the field required to process the request. The field may be an agriculture or horticulture.

The IoT sensor data of the set of inputs includes a soil moisture, a soil temperature, and an air temperature.

The one or more crop characteristics of the set of inputs includes a crop root zone depth, a crop age, a crop coefficient, a percentage crop canopy cover, a crop specific wilting point moisture content, and a crop specific permanent wilting point moisture content.

The one or more agro-meteorological weather data of the field includes a temperature, a relative humidity, a wind speed, a wind direction, an rainfall information, and a solar radiation information.

The one or more satellite earth data covering the field includes multi-spectral satellite data of Sentinel 2 or Landsat 8 or Landsat 9 satellite, and synthetic aperture radar satellite data from sensors of satellite like sentinel 1.

The one or more soil properties of the field includes a soil type, a soil depth, a wilting point moisture content, a permanent wilting point moisture content, a soil porosity, and a level of water table.

The irrigation system parameters includes but not limited to type of irrigation such as surface, drip, sprinkler, micro-sprinkler, sub-surface, spacing of emitters and emitter discharge, and crop row spacing.

The historical irrigation data includes but not limited to the day of irrigation, an amount of irrigation or time of irrigation pump operation with pump capacity and irrigation details.

The weather forecast data includes but not limited to an air temperature, a relative humidity, a precipitation (rain and snow), a solar radiation and a wind speed.

At step 404 of the method 400, the one or more hardware processors is configured to estimate using the set of inputs (i) a reference crop evapotranspiration (ETₒ) using the one or more agro-meteorological weather data and the IoT sensor data (ii) a crop ET under standard conditions (ET_{c}) using a crop coefficient (iii) a crop ET under non-standard conditions (ETₚₘ) using the crop coefficient and a soil factor (iv) a remote sensing based evapotranspiration (ETᵣₛ) using the one or more satellite earth observation data covering the field and (v) a field evapotranspiration ET (ET_{field}). Here, the ET field is an ensemble of the remote sensing based evapotranspiration and the crop ET under non-standard conditions.

For the above example, the set of inputs are received, to estimate the reference crop evapotranspiration (ETₒ) using the one or more agro-meteorological weather data and the IoT sensor data. The reference crop (ETₒ) is the evapotranspiration rate from a reference surface, not short of water. The reference surface is a hypothetical grass reference crop with specific characteristics as denoted in Equation 1,

$$Climate ( Radiation , Temperature , Wind Speed , Humidity ) + Grass Reference Crop ( well watered grass ) = ETo \qquad Equation 1$$

The crop evapotranspiration (ET) under standard conditions (ET_{c}) is estimated using the crop coefficient. The crop ET under standard conditions disease-free, well-fertilized crops, grown in large fields, under optimum soil water conditions, achieving full production under the given climatic conditions as in Equation 2,

$${ET}_{c} * {K}_{c} = {ET}_{c} , \qquad Equation 2$$

Further, the crop ET under non-standard conditions (ETₚₘ) is estimated using the crop coefficient and a soil factor. The crop ET is grown under management and environmental conditions that differ from the standard conditions. When cultivating crops in fields deviate from ET_{c} due to non-optimal conditions such as the presence of pests and diseases, soil salinity, low soil fertility, water shortage or waterlogging. This may result in scanty plant growth, low plant density and may reduce the ET rate below ET_{c} as defined in Equation 3,

$${ET}_{o} * ( {K}_{s} * {K}_{c} ) = {ET}_{c} adj \qquad Equation 3$$

Where, K₃=f {biotic stress, abiotic stress}
   The extent of biotic stress (due to pest/disease attack) is calculated using the field level smartphone camera images. The abiotic stress (due to temperature, moisture, etc.) is calculated using remote sensing-based vegetation indices such as Normalized Difference Vegetation Index (NDVI), Normalized Difference Water Index (NDWI) and Leaf Chlorophyll Index (LCI).

Then, the remote sensing-based ET (ETᵣₛ) is estimated using the one or more satellite earth data covering the field. Satellite based earth observations such as multi-spectral and thermal infra-red region are used to calculate the ETᵣₛ.

Finally, the field level ET (ET_{field}) is estimated using the ensemble based approach Penman-Montheith based ET (ETₚₘ) and the remote sensing based ET (ETᵣₛ) are used as input and filtering techniques are used to calculate the field level ET (ET_{field}) as in Equation 4,

$${ET}_{field} = f \{ {ET}_{pm} , {ET}_{rs} \} \qquad Equation 4$$

At step 406 of the method 400, the one or more hardware processors is configured to estimate a runoff using a curve number method based on the one or more agro-meteorological weather, and the one or more satellite earth data covering the field.

Further for the above example, the runoff is calculated using Soil Conservation System (SCS) curve number method. Where land cover condition estimated using satellite based land use land cover classification and antecedent rainfall observed by weather station are used as input as defined in Equation 5,

$$Q = \frac{{( P - {I}_{a} )}^{2}}{( P - {I}_{a} ) + S} \qquad Equation 5$$

Where, Q=Runoff (in), P=rainfall (in), S=potential maximum retention after runoff begins, Iₐ=initial abstractions Iₐ=0.2×S.
Subsetting the Iₐ value into Q to obtain the Equation 6 and 7,

$$Q = \frac{{( P - 0.2 S )}^{2}}{( P + 0.8 S )} \qquad Equation 6$$

$$\begin{matrix}( SCS Curve Number Method ) & \\ S = \frac{1000}{CN} - 10 & Equation 7\end{matrix}$$

Now step 408 of the method 400, the one or more hardware processors is configured to calculate a soil water balance of the field using the field level evapotranspiration (ET_{field}), the runoff, the effective rainfall information, the IoT sensor data, the one or more crop characteristics and the one or more soil properties.

In one embodiment, the soil water balance approach is defined using conceptual multi-layered horizontal soil profile and a vertical soil profile. The water storage in different layers of the soil is governed by various factors such as the effective rainfall, the irrigation, the evapotranspiration, the runoff, the percolation, the deep percolation and, the capillary rise. The storage of soil moisture (Δₛ) is a function of a first set of factors and a second set of factors as in Equation 8,

$${Δ}_{s} = the first set of factors ( input ) - the second set of factors ( output ) \qquad Equation 8$$

The first set of factors includes the effective rainfall, an irrigation, capillary rise, the runoff, a percolation, a deep percolation, and the evapotranspiration. The effective rainfall is obtained from the weather station observation and converted to effective rainfall (i.e., amount of water that enters the soil). Irrigation is calculated using the details of irrigation system. Capillary rise is calculated using a linear model and a soil saturation percentage. The second set of factors includes the runoff, the percolation is a component of the effective rainfall, and the deep percolation is a component of the vertical soil water movement. The runoff is calculated using the SCS curve number method as in Equation 5. The percolation is a component of the effective rainfall that defines a vertical movement of water into the soil profile. Deep percolation is a component of the vertical soil water movement where the water is lost to the subsurface and never recovered by natural process such as the capillary rise. Evapotranspiration is calculated using multiple methods such as Penman-Monteith Equations 1-3 and a METRIC model.

In one embodiment, the water balance equations are described below,

$$Total Available Water ( TAW ) = ( FC - PWP ) * Root zone depth for that crop growth stage \qquad Equation 9$$

Where, Fc=field capacity and PWP=permanent wilting point
Further, the readily available water is a sum of the total available water and depletion coefficient as described in Equation 10,

$$Readily Available Water ( RAW ) = TAW * Depletion coefficient \qquad Equation 10$$

The change in storage is calculated in Equation 11,

$$DS = P + I - {E}_{TC} - R \qquad Equation 11$$

Where, DS=Storage in soil profile also refereed as RAW,
P=Cumulative rainfall (mm), I=Irrigation applied (mm), E_{Tc}=Crop Evapotranspiration, R=Runoff.
The available soil profile moisture (APM) in Equation 12,

$${APM}_{i - 1} + {DS}_{i} ( For 1 st day to start the balance {APM}_{i - 1} = TAW ) \qquad Equation 12$$

Decide if i^{th} day is the irrigation day in Equation 13,

$${Check}_{i} {APM}_{i} = ( TAW - ( RAW - 2 ) ) \qquad Equation 13$$

The APM is calculated for each day till APM₍ᵢ₎ reaches (TAW−(RAW-2)) and irrigation irrigation amount is calculated in mm as in Equation 14,

$$IR = \frac{TAW - APM ( i )}{Irrigation System Efficiency} \qquad Equation 14$$

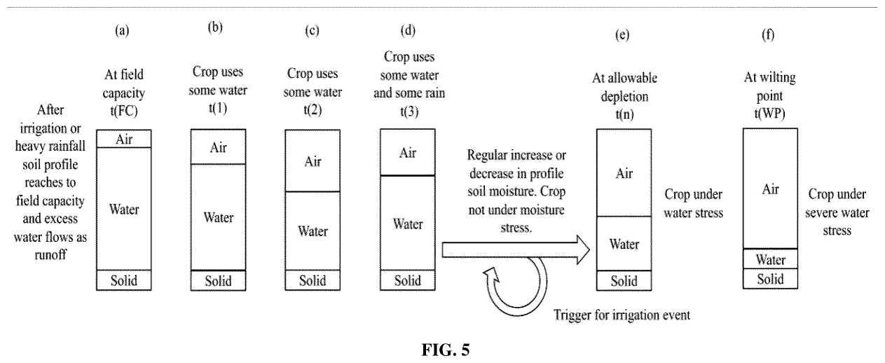

In one embodiment, Irrigation scheduling considers entire soil profile. The soil profile comprises of solid, air and water, where water is absorbed by roots for consumptive use and empty space is filled by air. FIG. 5 represents variation of water in soil profile at different time instances where (1) after irrigation, (2) during crop using soil moisture for metabolism, (3) addition of soil moisture due to rainfall, (4) soil profile reaching set allowable depletion, (5) soil profile reaching wilting point. In normal situation profile moisture varies between instance as described above (1) to (4). After stage (5) crop goes under water stress, the severity of water stress increases after wilting point after stage (5). The system and method described in this invention simulates soil profile to calculate the available soil moisture.

The amount of water required to fill the air space is calculated by soil water balance approach. The flowchart depicts Irrigation scheduling parameters such as irrigation interval, Depth of irrigation, Percent allowable depletion (based on depletion coefficient). The output of ensemble-based soil moisture estimation is used as input for irrigation scheduling. The profile soil moisture is calculated at daily interval and irrigation event is triggered based on the condition available profile moisture is less than percent allowable depletion. Once a day is identified as irrigation day the parameters specific to field and irrigation system are used and volume of water required, and time of irrigation is calculated.

### Example Irrigation System Parameters

Drip—Emitter spacing, lateral spacing, Emitter discharge, weighted fraction (e.g., assumed to be 0.4 as default), System efficiency (e.g., assumed to be 90 as default).
Drip: No of Emitters N=(Drip Spacing*Lateral Spacing)/Plot Area Sprinkler or micro Sprinkler−Sprinkler spacing, lateral spacing, Nozzle discharge, System efficiency (e.g., assumed to be 70 or 75%), weighted fraction (e.g., assumed to be 0.7 or 0.8 or 1 as options).
Sprinkler or micro Sprinkler: No of Emitters N=(Sprinkler Spacing*Length of pipeline) or plot area.
Surface—Size of Pump (Pump Discharge), Water Conveyance Efficiency (Pipe and water channel).
Surface: No of Emitters N=1 (as surface irrigation as one outlet, if required add outlets)

$$Volume of Water to Irrigate V = Weighted Fraction * Plot Area * IR Time = V / ( N * Emitter Discharge )$$

Note: 1. Weighted Fraction is different for irrigation type such as Drip or Sprinkler or Surface irrigation. 2. Irrigation Requirement differs for surface conditions i.e., IR or IR_{SEL} or IR_{SCC}.

Now step 410 of the method 400, the one or more hardware processors is configured to estimate the one or more soil properties and the one or more crop characteristics by using an inverse modelling approach, wherein the one or more soil properties includes a field capacity, and the one or more crop characteristics includes the crop coefficient.

The inverse modelling is applied for estimation of the field capacity and the crop coefficient using the soil moisture values. In forward model profile soil moisture is calculated using the soil water balance showed in Equation 9, 10 and 11.

However, the soil moisture values can be easily observed using soil moisture sensors or traditional gravimetric methods. The soil moisture storage in the soil profile and depletion coefficient are utilized to trigger an irrigation event feedback comprising a time of irrigation in the field. The process of inversion modelling where observed in the soil moisture values at multiple time periods are used to calculate the field capacity and the crop coefficient. The simplified soil water balance model for inverse model is described in Equation 15,

$$RAW = Irrigation + Rainfall - Runoff - Percolation + Capillary Rise - Deep Percolation - Crop Evapotranspiration \qquad Equation 15$$

The crop evapotranspiration uses energy balance method for ET and range of crop coefficient values (e.g., 0.8, 0.9, 1.0, 1.1, 1.2, etc.). The APM/RAW is compared with in-situ soil moisture values as below,
If [RAWₜ₁−(Root Zone Depth×Soil Moisture)ₜ₁]→0 (very close to zero + or −ve)
AND [RAWₜ₂−(Root Zone Depth×Soil Moisture)ₜ₂]→0 (very close to zero + or −ve)
   Then, the value of field capacity and the crop coefficient is selected. The entire assessment is performed for the vertical zone (Homogeneous portion of the field or region of interest). The values of the field capacity and the crop coefficient are then used for daily soil water balance. The steps of inverse modeling includes below in Table 1,

TABLE 1
Inverse modelling

Calculate the effective rainfall, the runoff for day 1 and 1 + n
Calculate the ET using PM for day 1 and 1 + n (using different values
of crop coefficient)
Calculate the percolation, the deep percolation, the capillary rise for
day 1 and 1 + n
Calculate the soil moisture available in the soil profile using the soil
water balance for day 1 and 1 + n (using different values of field
capacity)
Collect in-situ observation of soil moisture (sensor or gravimetric
method)
Compare the estimated soil moisture with in-situ soil moisture and
identify the closest match for day 1 and 1 + n
Select the field capacity and the crop coefficient for the close match
of soil moisture.
Utilizing the forward model with the selected field capacity and crop
coefficient and continue the soil water balance.

At step 412 of the method 400, the one or more hardware processors is configured to estimate an ensemble-based field soil moisture using the soil water balance, and a soil moisture estimated using the IoT sensor data and a soil moisture estimated using a synthetic aperture radar satellite data.

The field soil moisture is a filtering technique estimated using the soil moisture observed using the IoT sensor data, the soil moisture observed using the synthetic aperture radar satellite data, and the soil moisture sensor as described in Equation 1,

$$Field soil moisture = f \{ soil {moisture}_{ET} , Soil {moisture}_{SAR} , Soil {moisture}_{sensor} \} \qquad Equation 16$$

Referring now FIG. 6, the field soil moisture is estimated using a horizontal and vertical soil profile which is a function of a first set of factors and a second set of factors. The first set of factors includes the effective rainfall, an irrigation, capillary rise, the runoff, the percolation, the deep percolation, and the evapotranspiration. The second set of factors includes the runoff, the percolation is a component of the effective rainfall, and the deep percolation is a component of the vertical soil water movement. the soil moisture is estimated for a horizontal soil profile and a vertical soil profile.

At step 414 of the method 400, the one or more hardware processors is configured to estimate crop water requirement of the field using (i) the field soil moisture and (ii) a plurality of irrigation scheduling parameters comprising an irrigation interval, a depth of irrigation, a percent allowable depletion based on depletion coefficient. The crop water requirement of the field is estimated using the irrigation data, the effective rainfall information, the runoff, a percolation, capillary rise, deep percolation, and the reference crop evapotranspiration (ETₒ).

FIG. 5 depicts variation of air and water in soil profile at various time steps using the system of FIG. 2, in accordance with some embodiments of the present disclosure, in accordance with some embodiments of the present disclosure.

FIG. 6 depicts soil water balance using the system of FIG. 2, in accordance with some embodiments of the present disclosure, in accordance with some embodiments of the present disclosure.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments of present disclosure herein addresses unresolved problem of water requirement. The embodiment, thus provides system and method for estimating crop water requirement using multi-sensor data fusion. Moreover, the embodiments herein further estimates crop water requirement for scheduling irrigation or horticulture activity. The method includes observations from agro-meteorological stations, field level sensors, weather forecast and satellite based earth observations in multi-spectral and synthetic aperture radar observations.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g., any kind of computer like a server or a personal computer, or the like, or any combination thereof. The device may also include means which could be e.g., hardware means like e.g., an application-specific integrated circuit (ASIC), a field-programmable gate array (FPGA), or a combination of hardware and software means, e.g., an ASIC and an FPGA, or at least one microprocessor and at least one memory with software processing components located therein. Thus, the means can include both hardware means and software means. The method embodiments described herein could be implemented in hardware and software. The device may also include software means. Alternatively, the embodiments may be implemented on different hardware devices, e.g., using a plurality of CPUs.

The embodiments herein can comprise hardware and software elements. The embodiments that are implemented in software include but are not limited to, firmware, resident software, microcode, etc. The functions performed by various components described herein may be implemented in other components or combinations of other components. For the purposes of this description, a computer-usable or computer readable medium can be any apparatus that can comprise, store, communicate, propagate, or transport the program for use by or in connection with the instruction execution system, apparatus, or device.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

What is claimed is:

1. A processor-implemented method for estimating crop water requirement, the method comprising:
receiving from a field via one or more hardware processor a set of inputs comprising a IoT sensor data, one or more crop characteristics, one or more agro-meteorological weather data of the field, one or more satellite earth observation data covering the field, one or more soil properties of the field, a historical irrigation data, and a weather forecast data,
wherein the one or more agro-meteorological weather data includes a temperature, a relative humidity, a wind speed, a wind direction, an effective rainfall information, and a solar radiation information,
wherein the IoT sensor data includes a soil moisture, a soil temperature, and an air temperature;
estimating using the set of inputs via the one or more hardware processors (i) a reference crop evapotranspiration (ET) using the one or more agro-meteorological weather data and the IoT sensor data (ii) a crop ET under standard conditions (ET_{c}) using a crop coefficient (iii) a crop ET under non-standard conditions (ETₚₘ) using the crop coefficient and a soil factor (iv) a remote sensing based evapotranspiration (ETᵣₛ) using the one or more satellite earth observation data covering the field and (v) a field evapotranspiration ET (ET_{field});
estimating a runoff via the one or more hardware processors using a curve number method based on the one or more agro-meteorological weather, and the one or more satellite earth observation data covering the field;
calculating a soil water balance of the field via the one or more hardware processors using the field level evapotranspiration (ET_{field}), the runoff, the effective rainfall information, the IoT sensor data, the one or more crop characteristics and the one or more soil properties;
estimating via the one or more hardware processors the one or more soil properties and the one or more crop characteristics by using an inverse modelling approach, wherein the one or more soil properties includes a field capacity, and the one or more crop characteristics includes the crop coefficient;
estimating an ensemble-based field soil moisture via the one or more hardware processors using the soil water balance, and a soil moisture estimated using the IoT sensor data and a soil moisture estimated using a synthetic aperture radar satellite data; and
estimating via the one or more hardware processors crop water requirement of the field using (i) the field soil moisture and (ii) a plurality of irrigation scheduling parameters comprising an irrigation interval, a depth of irrigation, a percent allowable depletion based on depletion coefficient.

2. The processor-implemented method as claimed in claim 1, wherein the crop water requirement of the field is estimated using the irrigation data, the effective rainfall information, the runoff, a percolation, capillary rise, deep percolation, and the reference crop evapotranspiration (ETₒ).

3. The processor-implemented method as claimed in claim 1, wherein the field soil moisture value is estimated using a process-based soil moisture sensor, the soil moisture observed using the synthetic aperture radar satellite data, and the soil moisture observed using the IoT sensor data.

4. The processor-implemented method as claimed in claim 3, wherein a filtering technique is applied over the field soil moisture value to correct forthcoming soil field moisture values avoiding drastic increase or decrease of the soil moisture values, and
wherein the soil moisture is estimated for a horizontal soil profile and a vertical soil profile.

5. The processor-implemented method as claimed in claim 1, wherein the inverse modelling is applied for estimation of the field capacity and the crop coefficient of the field using the field soil moisture values, wherein the soil moisture storage in the soil profile and depletion coefficient are utilized to trigger an irrigation event feedback comprising a time of irrigation in the field, and wherein the crop water requirement for consecutive day is estimated based on the irrigation event feedback.

6. A system, for estimating crop water requirement comprising:
a memory storing instructions;
one or more communication interfaces; and
one or more hardware processors coupled to the memory via the one or more communication interfaces, wherein the one or more hardware processors are configured by the instructions to:
receive from a field a set of inputs comprising a IoT sensor data, one or more crop characteristics, one or more agro-meteorological weather data of the field, one or more satellite earth observation data covering the field, one or more soil properties of the field, a historical irrigation data, and a weather forecast data,
wherein the one or more agro-meteorological weather data includes a temperature, a relative humidity, a wind speed, a wind direction, an effective rainfall information, and a solar radiation information,
wherein the IoT sensor data includes a soil moisture, a soil temperature, and an air temperature;
estimate using the set of inputs (i) a reference crop evapotranspiration (ET) using the one or more agro-meteorological weather data and the IoT sensor data (ii) a crop ET under standard conditions (ET_{c}) using a crop coefficient (iii) a crop ET under non-standard conditions (ETₚₘ) using the crop coefficient and a soil factor (iv) a remote sensing based evapotranspiration (ETᵣₛ) using the one or more satellite earth observation data covering the field and (v) a field evapotranspiration ET (ET_{field});
estimate a runoff using a curve number method based on the one or more agro-meteorological weather, and the one or more satellite earth observation data covering the field;
calculate a soil water balance of the field using the field level evapotranspiration (ET_{field}), the runoff, the effective rainfall information, the IoT sensor data, the one or more crop characteristics and the one or more soil properties;
estimate the one or more soil properties and the one or more crop characteristics by using an inverse modelling approach, wherein the one or more soil properties includes a field capacity, and the one or more crop characteristics includes the crop coefficient;
estimate an ensemble-based field soil moisture using the soil water balance, and a soil moisture estimated using the IoT sensor data and a soil moisture estimated using a synthetic aperture radar satellite data; and
estimate crop water requirement of the field using (i) the field soil moisture and (ii) a plurality of irrigation scheduling parameters comprising an irrigation interval, a depth of irrigation, a percent allowable depletion based on depletion coefficient.

7. The system as claimed in claim 6, wherein the crop water requirement of the field is estimated using the irrigation data, the effective rainfall information, the runoff, a percolation, capillary rise, deep percolation, and the reference crop evapotranspiration (ETₒ).

8. The system as claimed in claim 6, wherein the field soil moisture value is estimated using a process-based soil moisture sensor, the soil moisture observed using the synthetic aperture radar satellite data, and the soil moisture observed using the IoT sensor data.

9. The system as claimed in claim 8, wherein a filtering technique is applied over the field soil moisture value to correct forthcoming soil field moisture values avoiding drastic increase or decrease of the soil moisture values, and
wherein the soil moisture is estimated for a horizontal soil profile and a vertical soil profile.

10. The system as claimed in claim 6, wherein the inverse modelling is applied for estimation of the field capacity and the crop coefficient of the field using the soil field moisture values, wherein the soil moisture storage in the soil profile and depletion coefficient are utilized to trigger an irrigation event feedback comprising a time of irrigation in the field, and
wherein the crop water requirement for consecutive day is estimated based on the irrigation event feedback.

11. One or more non-transitory machine-readable information storage mediums comprising one or more instructions which when executed by one or more hardware processors cause:
receive from a field a set of inputs comprising a IoT sensor data, one or more crop characteristics, one or more agro-meteorological weather data of the field, one or more satellite earth observation data covering the field, one or more soil properties of the field, a historical irrigation data, and a weather forecast data,
wherein the one or more agro-meteorological weather data includes a temperature, a relative humidity, a wind speed, a wind direction, an effective rainfall information, and a solar radiation information,
wherein the IoT sensor data includes a soil moisture, a soil temperature, and an air temperature;
estimate using the set of inputs (i) a reference crop evapotranspiration (ET) using the one or more agro-meteorological weather data and the IoT sensor data (ii) a crop ET under standard conditions (ET_{c}) using a crop coefficient (iii) a crop ET under non-standard conditions (ETₚₘ) using the crop coefficient and a soil factor (iv) a remote sensing based evapotranspiration (ETᵣₛ) using the one or more satellite earth observation data covering the field and (v) a field evapotranspiration ET (ET_{field});
estimate a runoff using a curve number method based on the one or more agro-meteorological weather, and the one or more satellite earth observation data covering the field;
calculate a soil water balance of the field using the field level evapotranspiration (ET_{field}), the runoff, the effective rainfall information, the IoT sensor data, the one or more crop characteristics and the one or more soil properties;
estimate the one or more soil properties and the one or more crop characteristics by using an inverse modelling approach, wherein the one or more soil properties includes a field capacity, and the one or more crop characteristics includes the crop coefficient;
estimate an ensemble-based field soil moisture using the soil water balance, and a soil moisture estimated using the IoT sensor data and a soil moisture estimated using a synthetic aperture radar satellite data; and
estimate crop water requirement of the field using (i) the field soil moisture and (ii) a plurality of irrigation scheduling parameters comprising an irrigation interval, a depth of irrigation, a percent allowable depletion based on depletion coefficient.

12. The one or more non-transitory machine-readable information storage mediums of claim 11, wherein the crop water requirement of the field is estimated using the irrigation data, the effective rainfall information, the runoff, a percolation, capillary rise, deep percolation, and the reference crop evapotranspiration (ETₒ).

13. The one or more non-transitory machine-readable information storage mediums of claim 11, wherein the field soil moisture value is estimated using a process-base soil moisture sensor, the soil moisture observed using the synthetic aperture radar satellite data, and the soil moisture observed using the IoT sensor data.

14. The one or more non-transitory machine-readable information storage mediums of claim 13, wherein a filtering technique is applied over the field soil moisture value to correct forthcoming soil field moisture values avoiding drastic increase or decrease of the soil moisture values, and wherein the soil moisture is estimated for a horizontal soil profile and a vertical soil profile.

15. The one or more non-transitory machine-readable information storage mediums of claim 11, wherein the inverse modelling is applied for estimation of the field capacity and the crop coefficient of the field using the field soil moisture values, wherein the soil moisture storage in the soil profile and depletion coefficient are utilized to trigger an irrigation event feedback comprising a time of irrigation in the field, and wherein the crop water requirement for consecutive day is estimated based on the irrigation event feedback.
